(19) United States
(12) Patent Application Publication
(10) Pub. No.: US 2007/0049773 A1
(43) Pub. Date: Mar. 1, 2007

(54) Process for functionalising a phenolic compound carrying an electron-donating group

(76) Inventors: Charles Mioskowski, Strasbourg (FR); Alain Wagner, Lyon (FR); Nicolas Bensel, Pfastatt (FR); Virginie Pevere, Lyon (FR); Jean-Roger Desmurs, St-Symphorien D'Ozon (FR)

Correspondence Address: Rhodia Inc.;Legal Department, CN 7500, 8 CEDAR BROOK DRIVE, CRANBURY, NJ, 08512-7500, US

(21) Appl. No.: 11/592,930

(22) Filed: Nov. 3, 2006

Related U.S. Application Data
(63) Continuation of application No. 10/262,702, filed on Oct. 2, 2002.
Application No. 10/262,702 is a continuation of application No. 09/529,258, filed on Aug. 11, 2000, now abandoned.

(30) Foreign Application Priority Data
Oct. 8, 1997 (FR) 97/12548

(51) Int. Cl. C07C 39/24; C07C 39/34; C07C 45/90

(52) U.S. Cl. 568/426; 568/774

(57) ABSTRACT

The invention concerns a method for functionalizing a phenolic compound bearing an electron-donor group, in said group para position, inter alia a method for the amidoalkylation of a phenolic compound bearing an electron-donor group, and more particularly, a phenolic compound bearing an electron-donor group preferably, in the hydroxyl group ortho position. The method for functionalizing in para position with respect to an electron-donor group carried by a phenolic compound is characterised in that the phenolic compound bearing an electron-donor group is subjected to the following steps: a first step which consists of protecting the hydroxyl group in the form of a sulphonic ester function; a second step which consists in reacting the protected phenolic compound with an electrophilic reagent; optionally, a third step deprotecting the hydroxyl group.

The present invention relates to a process for functionalising a phenolic compound carrying an electron-donating group in the position para to said group.

The invention also relates to a process for amidoalkylation of a phenolic compound carrying an electron-donating group.

More particularly, the invention relates to a phenolic compound carrying an electron-donating group, preferably in the position ortho to a hydroxy group.

In the remainder of the description, the term “phenolic compound” means any aromatic compound carrying at least one hydroxy group and the term “aromatic” denotes the conventional concept of aromaticity as defined in the literature, in particular by Jerry MARCH, “Advanced Organic Chemistry”, 4^{th} edition, John Wiley & Sons, 1992, pp 40 ff.

The phenolic compound used in the process of the invention is a phenolic compound carrying an electron-donating group, preferably in the position ortho to the hydroxy group and having a hydrogen atom in the position para to the electron-donating group.

In the present text, the term “electron-donating group” means a group as defined by H. C. BROWN in the work by Jerry MARCH, “Advanced Organic Chemistry”, 4^{th} edition, John Wiley & Sons, 1992, Chapter 9, pp. 273-292.

Amidoalkylation of a phenolic compound carrying an electron-donating poses a problem to the skilled person.

Thus in the more particular case of amidomethylating guaiacol (2-methoxyphenol), the reaction is more particularly carried out at the position 4—to the hydroxy group.

The aim of the present invention is to provide a process for carrying out preferential amidoalkylation, more particularly, amidomethylation in the position 4—with respect to a methoxy group.

During the course of its research, the Applicant has discovered that the process can be applied not only to amidoalkylation reactions but also to all electrophilic substitution reactions which involve an electrophilic reactant which comprises at least one electrophilic carbon atom C, i.e., a carbon atom with an electron deficit, or an electrophilic sulphur atom.

The present invention provides a process for functionalisation in the position para with respect to an electron-donating group carried by a phenolic compound, characterized in that the phenolic compound carrying an electron-donating group undergoes the following steps:
a first step which consists of protecting the hydroxy group by means of a sulphonic ester function;
a second step which consists of reacting the protected phenolic compound with an electrophilic reactant;
optionally, a third step of deprotecting the hydroxy group.

It has been discovered that it is possible to introduce a group comprising an electrophilic carbon atom or a sulphur atom into the position para to the electron-donating group carried by the phenolic compound, provided that the hydroxy group is protected by a sulphonic ester function.

The process of the invention can be applied to all electrophilic substitution reactions, in particular amidoalkylation reactions, Friedel-Crafts reactions, in particular acylation, sulphonylation, alkylation, aminoalkylation reactions, hydroxyalkylation reactions, formylation reactions, carboxylation reactions and the like.

Protection of Hydroxy Group

In accordance with the process of the invention, the hydroxy group in the starting phenolic compound is protected in a first step.

The starting compound is a phenolic group carrying at least one electron-donating group, preferably in the position ortho to the hydroxy group and where the position para to the electron-donating group is free of substituents.

More particularly, it has general formula (I):

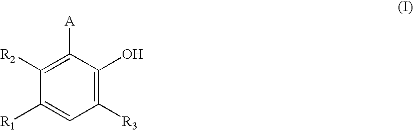

in which formula (I):
A represents an electron-donating group;
R₁, R₂ and R₃, which may be identical or different, represent:
   a hydrogen atom;
   a linear or branched alkyl radical containing 1 to 20 carbon atoms, optionally carrying one or more halogen atoms;
   a cycloalkyl group containing 3 to 8 carbon atoms, preferably 6 carbon atoms;
   an aralkyl radical containing 6 to 20 carbon atoms;
   an aryl radical containing 6 to 20 carbon atoms:
   a halogen atom;
   an electron-attracting group;
two groups R₁ and R₂ together with the two atoms carrying them can form a saturated, unsaturated or aromatic carbocyclic cycle containing 3 to 8 carbon atoms.

In formula (I), groups R₁, R₂ and R₃ can represent an electron-attracting group.

The term “electron-attracting group” means a group as defined by H. C. BROWN in the book by Jerry MARCH “Advanced Organic Chemistry”, 4^{th} edition, John Wiley & Sons, 1992, Chapter 9, pp. 273-292.

It is preferably a carboxy or an ester group (preferably containing 3 to 8 carbon atoms) or a nitrile group or a nitro group.

Preferred compounds are those with formula (I) in which R₁, R₂ and R₃ represent a hydrogen atom, a linear or branched alkyl group containing 1 to 4 carbon atoms, a halogen atom or a trifluoromethyl group.

Starting compounds with formula (I) include those with formula (I) where A represents one of the following groups:
a linear or branched alkyl group preferably containing 1 to 6 carbon atoms, more preferably containing 1 to 4 carbon atoms;
a cycloalkyl group containing 3 to 8 carbon atoms, preferably 6 carbon atoms;
the phenyl radical;
the benzyl or phenylethyl radical;
the hydroxy group;
a fluorine atom;
an alkoxy group containing 1 to 6 carbon atoms, more preferably 1 to 4 carbon atoms in the alkyl portion, or a phenoxy group;
an amino group, preferably di-substituted in which the substituents, which may be identical or different, are linear or branched alkyl radicals containing 1 to 6 carbon atoms, more preferably 1 to 4 carbon atoms, or a phenyl group;
an alkylamide or arylamide group in which the alkyl group contains 1 to 6 carbon atoms, preferably 1 to 4 carbon atoms, and the aryl group contains 6 to 12 carbon atoms and is preferably a phenyl group.

Preferred compounds have formula (I) in which A represents a methoxy or an ethoxy group.

The process of the invention consists in a first step of protecting the hydroxy function with a protecting group. To this end, the hydroxy function is transformed into a sulphonic ester.

Preferred sulphonic ester groups are:
tosylates (p-toluenesulphonates) —OSO₂—C₆H₄—CH₃;
brosylates (p-bromobenzenesulphonates) —OSO₂—C₆H₄—Br;
nosylates (p-nitrobenzenesulphonates) —OSO₂—C₆H₄—NO₂;
mesylates (methanesulphonates) —OSO₂—CH₃;
betylates (ammonioalkanesulphonates) —OSO₂—(CH₂)ₙNMe₃+where n is in the range 0 to 6;
triflates (trifluoromethanesulphonates) —OSO₂—CF₃;
nonaflates (nonafluorobutanesulphonates) —SO₂—C₄F₉;
tresylates (2,2,2-trifluoroethanesulphonates) —OSO₂—CHH₂—CF₃.

Thus the protected phenolic compound has general formula (II):

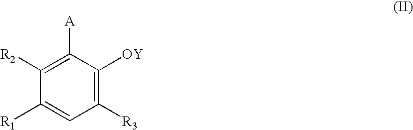

in which formula (II): 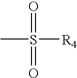
Y represents the following group: 4/28
   where R₄ represents a hydrocarbon radical containing 1 to 20 carbon atoms, more particularly:
   an alkyl radical containing 1 to 10 carbon atoms, preferably 1 to 4 carbon atoms, more preferably a methyl or ethyl radical, optionally carrying a halogen atom, a CF₃ group or an ammonium group (N(R₅)₄, R₅, which may be identical or different, representing an alkyl radical containing 1 to 4 carbon atoms;
   a cycloalkyl radical containing 3 to 8 carbon atoms, preferably a cyclohexyl radical;
   an aryl radical containing 6 to 12 carbon atoms, preferably a phenyl radical optionally carrying an alkyl radical containing 1 to 10 carbon atoms, preferably 1 to 4 carbon atoms and more preferably a methyl or an ethyl radical, a halogen atom, a CF₃ group or a NO₂ group;
   a CX₃ group where X represents a fluorine, chlorine or bromine atom;
   a CF₂—CF₃ group;
A, R₁, R₂ and R₃ have the meanings given above.

A protected phenol with formula (II) can be obtained by reacting, in the presence of a base, a starting phenolic compound with formula (I) with a protecting agent with the following formula (III):

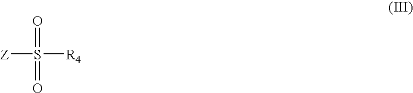

in which formula (III):
R₄ has the meaning given above;
Z represents:
   a hydroxy group or a halogen atom, preferably a chlorine or bromine atom.
   a —O—SO₂—R₄′ group where R₄′ which may be identical to or different from R₄, has the meaning given for R₄.

Preferred protecting agents have formula (III) where Z represents a chlorine or a bromine atom.

More particular preferred phenolic compounds used are:
pyrocatechin;
resorcin:
o-cresol;
m-cresol;
2-ethylphenol;
3-ethylphenol;
2-propylphenol;
2-sec-butylphenol;
2-tert-butylphenol;
3-tert-butylphenol;
2-methoxyphenol;
3-methoxyphenol;
2-ethoxyphenol;
3-ethoxyphenol;
2-chlorophenol;
3-chlorophenol;
2,3-dimethylphenol;
2,5-dimethylphenol;
2,6-dimethylphenol;
3,5-dimethylphenol;
vanillin;
2,3-dichlorophenol;
2,6-dichlorophenol;
3,5-dichlorophenol;
pyrogallol;
2,3,5-trimethylphenol;
2,3,6-trimethylphenol;
2,6-di-tert-butylphenol;
3,5-di-tert-butylphenol;
2,3,6-trichlorophenol;
1,2-dihydroxynaphthalene;
1,4-dihydroxynaphthalene;
1,5-dihydroxynaphthalene;
2,3-dihydroxynaphthalene;
2,6-dihydroxynaphthalene;
2,7-dihydroxynaphthalene;
4-methoxy-1-naphthol;
6-bromo-2-naphthol;
2-phenoxyphenol;
3-phenoxyphenol.

More particularly, the protecting agents used are:
triflic anhydride;
methanesulphonyl chloride;
trifluoromethanesulphonyl chloride;
benzenesulphonyl chloride;
p-toluenesulphonyl chloride.

A base, which can be a mineral or organic base, is used in the process of the invention with the aim of trapping the acidity.

Any type of base can be used.

The pKa of the base is generally more than 7.

The pKa is defined as the ionic dissociation constant of the acid/base couple, when water is used as the solvent.

To select a base with a pKa as defined by the invention, reference can, inter alia, be made to the “HANDBOOK OF CHEMISTRY AND PHYSICS, 66^{th} edition, pp D-161 and D-162.

Strong bases such as alkali metal hydroxides can be used, preferably sodium or potassium hydroxide or alkali metal salts, in particular alkali metal carbonates, bicarbonates, phosphates, hydrogen phosphates, sulphates, acetates and trifluoroacetates.

The concentration of the basic starting solution is not critical. The concentration of the alkali metal hydroxide solution used is generally in the range 10% to 50% by weight.

When the protecting agent is a halide, one preferred variation of the invention consists of adding a base, preferably a tertiary amine, to trap the liberated hydrogen acid.

Usable bases which can be cited include tertiary amines, more particularly those with general formula (IV):
N—(R₆)₃ (IV)

where:
groups R₆, which may be identical or different, represent hydrocarbon residues containing 1 to 20 carbon atoms, such as alkyl, cycloalkyl, aryl or heterocyclic groups;
two R₆ groups together With the nitrogen atom form a heterocycle containing 4 to 5 atoms.

More particularly:
symbols R₆ represent an alkyl radical containing 1 to 10 carbon atoms, preferably 1 to 4 carbon atoms, or a cyclopentyl or cyclohexyl or pyridinyl radical:
two R₆ groups together with the nitrogen atom for a piperidine or pyrrolidine cycle.

Examples of such amines which can be cited are triethylamine, tri-n-propylamine, tri-n-butylamine, methyldibutylamine, methyldicyclohexylamine, ethyldiisopropylamine, N,N-diethylcyclohexylamine, dimethylamino-4-pyridine, N-methylpiperidine, N-ethylpiperidine, N-n-butylpiperidine, 1,2-dimethylpiperidine, N-methylpyrrolidone and 1,2-dimethylpyrrolidine.

The phenolic compound and protecting agent are reacted in the presence of a base, preferably in an organic solvent.

Any type of polar or apolar organic solvent can be used, or a mixture of organic solvents.

An organic solvent which is inert under the reaction conditions can also be used. Thus the following can be used: aliphatic or cycloaliphatic hydrocarbons, which may or may not be halogenated, such as hexane, cyclohexane or dichloroethane, or aromatic hydrocarbons, which may or may not be halogenated, such as benzene, toluene, xylenes or chlorobenzenes: esters such as methyl benzoate, methyl terephthalate, methyl adipate, dibutyl phthalate; esters or ethers of polyols such as tetraethylene glycol diacetate; or aliphatic, linear or cyclic ethers such as isopropyl ether, tetrahydrofuran or dioxane; aliphatic or aromatic nitriles such as acetonitrile, propionitrile, butanenitrile, isobutanenitrile, benzonitrile or benzyl cyanide.

The different reactants and substrates are used in quantities which will be defined below.

The concentration of phenolic compound with formula (I) used in the solvent can fall within a wide range. In general, the concentration of the phenolic compound is in the range 1 to 5 mol/l, preferably close to 3 mol/l of solvent.

The quantity of base used, expressed as the ratio of the number of equivalents of OH⁻ to the number of moles of phenolic compound with formula (I), can fall within a wide range. Thus the OH⁻/phenolic compound mole ratio can be in the range 0.5 to 3.0, preferably in the range 1.0 to 2.0.

The quantity of protecting agent used, expressed as the ratio between the number of moles of protecting agent and the number of moles of starting phenolic compound, is advantageously in the range 1.0 to 1.5 and is preferably about 1.1.

The temperature at which the operation for protecting the hydroxy group of the phenolic compound is carried out is between 0° C. and ambient temperature (usually between 15° C. and 25° C.).

In one implementation of the invention, the reactants are mixed in any order.

A preferred implementation of the invention consists of adding the base to a reaction medium comprising the phenolic compound, an optional organic solvent, and the protecting agent.

At the end of the reaction, a phenolic compound is obtained which is protected which can be purified using conventional methods (liquid-liquid extraction, filtering).

Electrophilic substitution reactions

In accordance with the process of the invention, in a second step the electrophilic substitution reaction is carried out by bringing the protected phenolic compound into contact with an electrophilic reactant.

This step causes functionalisation by exchanging the hydrogen atom for a functional group comprising an electrophilic carbon atom or sulphur atom. This exchange is carried out specifically at the position para to the electron-donating group.

Exchange between the hydrogen atom and the electrophilic reactant can be carried out in the case of the following reactions: amidoalkylation reactions, Friedel-Crafts reactions, in particular acylation, sulphonylation, alkylation, aminoalkylation reactions, hydroxyalkylation reactions, formylation reactions, carboxylation reactions, etc. . . .

Amidoalkylation

The protected phenolic compound undergoes an amidoalkylation reaction. The expression “amidoalkylation” should be taken in its broadest sense as this term includes not only alkyl groups but also aralkyl groups.

In a subsequent step of the process of the invention, the protected phenolic compound, preferably with formula (II), is reacted (in the presence of a strong acid) with an amide or a carbamate and a carbonyl compound more particularly with general formula (V):

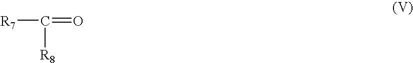

in which formula (V):
R₇ represents a hydrogen atom;
R₈ represents a hydrogen atom, a linear or branched alkyl radical containing 1 to 6 carbon atoms, a cycloalkyl radical containing 3 to 8 carbon atoms, an alkenyl radical containing 2 to 6 carbon atoms or a phenyl radical which may be substituted, or an electron-attracting group.

Examples of electron-attracting groups which are suitable for use in the present invention and which can be cited are:
a —CHO group;
a R₉—CO— or R₉—CO—(CH₂)ₘ— group where R₉ represents a linear or branched alkyl radical containing 1 to 6 carbon atoms and m represents a number from 1 to 3;
a —COOR₁₀ group in which R₁₀ represents a hydrogen atom or a linear or branched alkyl radical containing 1 to 6 carbon atoms;
a —CX₂H group where X represents a halogen atom, preferably a fluorine, chlorine or bromine atom;
a —CX₃ group where X represents a halogen atom, preferably a fluorine, chlorine or bromine atom;
a nitro group.

Examples of carbonyl compounds with formula (V) which can be cited are:
formaldehyde or a formaldehyde generator such as paraformaldehyde which can originate from an aqueous solution (methylene glycol) or used in the form of linear polyformaldehydes with any degree of polymerisation, preferably with a number of (CH₂O) motifs in the range 8 to 100 motifs;
glyoxal;
glyoxylic acid;
bromal;
fluoral.

Paraformaldehyde is the preferred carbonyl compound.

Said reactant is used either in the solid form or in the form of an aqueous solution with a concentration of less than 50% by weight, preferably in the range 20% to 50% by weight.

In accordance with the process of the invention, the protected phenolic compound is reacted with a carbonyl compound with formula (V) in the presence of an amide or a carbamate.

Examples of amides or carbamates which are suitable for use in the invention which can be cited are those which have the following formula (VI):

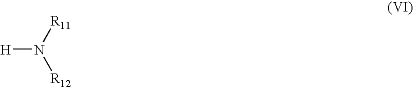

where:
at least one of groups R₁₁ and R₁₂ represents a group: 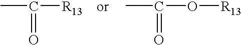
groups R₁₁, R₁₂ and R₁₃, which may be identical or different, represent:
   a hydrogen atom;
   a linear or branched alkyl radical containing 1 to 12 carbon atoms, optionally carrying halogen atoms;
   a cycloalkyl radical containing 3 to 8 carbon atoms, preferably a cyclopentyl radical, a cyclohexyl radical or a cycloheptyl radical;
   a cycloalkylalkyl radical, preferably cyclopentylalkyl, cyclohexylalkyl or cycloheptylalkyl where the alkyl portion contains 1 to 4 carbon atoms;
   an alkylaryl radical, preferably a phenylalkyl radical, where the alkyl portion contains 1 to 4 carbon atoms, preferably a benzyl radical;
two groups R₁₁ and R₁₂, together with the two atoms carrying them, can form a saturated, unsaturated or aromatic carbocylic cycle containing 3 to 8 carbon atoms.

Illustrative examples which can be cited for compounds with formula (VI) are acetamide, chloracetamide, benzamide and benzyl carbamate.

In the process of the invention, amidoalkylation of the protected phenolic compound is carried out in an acidic medium.

In accordance with the invention, a protonic acid with a pKa of 4.00 or less is used. More preferably, a protonic acid with a pKa of 3.00 or less is used.

The pKa is defined as the ionic dissociation constant of the acid/base couple when water is used as the solvent.

Selection of the acid with a pKa as defined by the invention can be made by referring, inter alia, to the “HANDBOOK OF CHEMISTRY AND PHYSICS”, 66^{th} edition, p. D-161 and D-162.

More particular examples of protonic acids which are suitable and which can be cited are halogenated or non halogenated mineral oxyacids such sulphuric acid, hydrochloric acid, chlorosulphonic acid, fluorosulphonic acid; phosphoric acids such as phosphoric acid, (2-ethylhexyl)phosphoric acid, (octylphenyl)phosphoric acid; phosphonic acids such as (2-ethylhexyl)(2-ethylhexyl)phosphonic acid; and perhalogenated or non perhalogenated carboxylic acids such as formic acid, citric acid, trichloroacetic acid, trifluoroacetic acid.

Halogenated or non halogenated sulphonic acids are also well suited to the present invention. These include fluorosulphonic acid, chlorosulphonic acid or trifluoromethanesulphonic acid, methanesulphonic acid, ethanesulphonic acid, ethanedisulphonic acid, benzenesulphonic acid, benzenedisulphonic acids, toluenesulphonic acids, naphthalenesulphonic acids, naphthalenedisulphonic acids and camphorsulphonic acids.

Preferably, sulphuric acid is used.

As in the previous step, the reaction is carried out in an organic solvent.

The organic solvents cited above are suitable but an aliphatic carboxylic acid is preferably used.

The carboxylic acid represents at least 50%, preferably 60% to 100%, of the weight of the organic solvents.

Any saturated monocarboxylic aliphatic acid which is liquid under the reaction conditions can be used, and preferably liquid at ambient temperature. The term “ambient temperature” generally means a temperature in the range 15° C. to 25° C.

In order to determine whether a saturated aliphatic monocarboxylic acid is suitable for use in the present invention, reference can be made to the literature, in particular to the “HANDBOOK OF CHEMISTRY AND PHYSICS”.

Examples of saturated aliphatic monocarboxylic acids which are suitable for use in the present invention which can be cited include acetic acid, propionic acid, butyric acid, isobutyric acid, pentanoic acid and 2-methylbutanoic acid.

Of all of the saturated aliphatic monocarboxylic acids, acetic acid is preferred.

Regarding the concentrations and quantities of the reactants to be used, preferred conditions are defined below.

In accordance with the process of the invention, the carbonyl compound with formula (V) can be reacted with the protected phenolic compound preferably with formula (II) in the presence of an amide or carbamate preferably with formula (VI).

The concentration of protected phenolic compound used in the solvent can fall within a wide range. In general, the concentration of phenolic compound is in the range 0.2 to 5.0 mol/l and is preferably in the range 0.2 to 2.0 mol/l of solvent.

Preferably, a quantity of carbonyl compound is selected which is in excess with respect to the protected phenolic compound.

The ratio between the number of moles of carbonyl compound and the number of moles of protected phenolic compound is at least 1, preferably in the range 1.0 to 2.0.

Regarding the quantity of amide or carbamate expressed with respect to the carbonyl compound, it is generally in excess. The ratio between the number of moles of amide or carbamate and the number of moles of carbonyl compound is advantageously selected so as to be in the range 1.0 to 3.0, preferably about 2.0.

The quantity of strong acid used is such that the mole ratio H⁻/protected phenolic compound can be in the range 1.0 to 20, preferably in the range 2.0 to 3.0.

The reaction is advantageously carried out between ambient temperature and 120° C. preferably in the range 40° C. to 80° C.

This amidoalkylation reaction is generally carried out at atmospheric pressure.

At the end of the reaction, an amidoalkylated product is obtained. It is in general in the form of a paste. The medium is diluted with any solvent (for example ethyl acetate), then a base is added, preferably sodium hydroxide or potassium hydroxide in a quantity such that the pH is in the range 7 to 10.

The amidoalkylated product is extracted with a suitable solvent, for example ethyl acetate and/or chloroform.

The product obtained can optionally be purified by re-crystallisation from a suitable solvent, for example chloroform or ethyl acetate.

At the end of this step, an amidoalkylated protected phenolic compound is obtained which preferably has formula (VII):

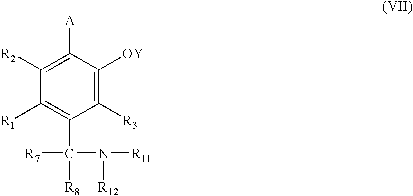

where the different symbols have the meanings given above.

Deprotection of Hydroxy Group

In accordance with the invention, in a final step, the sulphonyl group is cleaved using a base to liberate the hydroxy group.

Under the deprotection conditions, the process of the invention can produce a phenolic compound carrying an electron-donating group, and an amidoalkylated group or an alkylamine group in the position para to the electron-donating group.

To this end, a basic solution is used, preferably an aqueous sodium hydroxide solution, potassium hydroxide solution, or a sodium or potassium carbonate solution.

The quantity of base, expressed as the ratio between the number of moles of base and the number of moles of protected and amidoalkylated phenolic compound, is generally at least 2, preferably in the range 2 to 4.

This operation is preferably carried out in an organic solvent. The alcohols, preferably aliphatic alcohols and more particularly methanol, ethanol and isopropanol, are the solvents of choice.

The concentration of protected and amidoalkylated phenolic compound is advantageously in the range 0.5 to 1 mole/litre.

It is generally heated under reflex, for a period in the range 4 to 24 hours.

The solvent is eliminated by distillation.

After adding water, the product formed, namely the amidoalkylated and deprotected phenolic compound, is extracted after acidification, preferably at a pH of 6, in an organic solvent which is non miscible with water, such as dichloromethane or ethyl acetate.

The product is recovered after eliminating the reaction solvent.

In a further variation of the process of the invention, it is possible, not only during this operation, to selectively cleave the amide function and thus obtain an alkylamine function.

The amidoalkylated and protected product obtained at the end of the second step preferably with formula (VII), is reacted with a solution of a mineral acid.

The following strong acids are in particular used: hydrochloric acid, perchloric acid, to sulphuric acid or hydrobromic acid.

The quantity of acid, expressed as the ratio between the number of H⁺ ions and the number of moles of protected and amidoalkylated compound, is generally in the range 5 to 10.

The concentration of acid solution is not critical and a dilute or a concentrated acid solution can be used.

Heating is carried out as before, preferably at the reflux temperature.

A protected phenolic compound carrying an alkylamine group in the position para to the electron-donating group is recovered.

Friedel-Crafts Reactions: Acylation, Sulphonylation and Alkylation

In a variation of the process of the invention, the protected phenolic compound is reacted in Friedel-Crafts type reactions, in particular acylation, sulphonylation or alkylation reactions.

Regarding the acylation reactant, carboxylic acids and their halide or anhydride derivatives are used, preferably anhydrides.

Regarding the sulphonylation reactant, sulphonyl or aminosulphonyl halides or anhydrides are used in particular.

More particularly, the acylation or sulphonylation reactants have the following formulae:
in which formulae (VIII) or (IX): 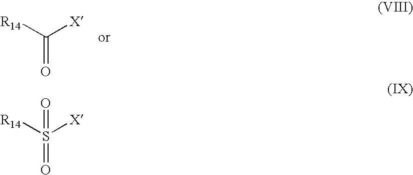
   R₁₄ represents a linear or branched, saturated or unsaturated aliphatic radical containing 1 to 24 carbon atoms; a saturated, unsaturated or aromatic, monocyclic or polycyclic cycloaliphatic radical containing 4 to 12 carbon atoms; or a linear or branched, saturated or unsaturated aliphatic radical carrying a cyclic substituent;
   X′ represents a halogen atom, preferably a chlorine or bromine atom;
in formula (VIII):
   X′ represents a —O—CO—R₁₅ radical where R₁₅, which may be identical to or different from R₁₄, has the same meaning as R₁₄;
in formula (IX):
   X′ represents a —O—SO₂—R₁₅ radical where R₁₅, which may be identical to or different from R₁₄, has the same meaning as R₁₄;
   R₁₄ represents:
      an R₁₆—O— alkoxy radical where R₁₆ has the same meaning as R₁₄;
      an amino group (R₁₇)(R₁₈)—N— where R₁₇, which may be identical to or different is from R₁₈, has the same meaning as R₁₄.

The term cyclic substituent” preferably means a saturated, unsaturated or aromatic carbocyclic cycle, preferably cycloaliphatic or aromatic, in particular cycloaliphatic containing 6 carbon atoms in the cycle, or benzenic.

More preferably, R₁₄ represents a linear or branched alkyl radical containing 1 to 12 carbon atoms, preferably 1 to 6 carbon atoms; the hydrocarbon chain can optionally be interrupted by a heteroatom (for example oxygen), by a functional group (for example —CO—) and/or can carry a substituent (for example a halogen or a CF₃ group).

R₁₄ preferably represents an alkyl radical containing 1 to 4 carbon atoms, such as methyl, ethyl, propyl, isopropyl, butyl, isobutyl, sec-butyl or tert-butyl.

Radical R₁₄ also preferably represents a phenyl radical which can optionally be substituted. The radical has to be more deactivated than the phenolic compound as if not, it would take part in acylation of the acylation agent itself.

More particular examples of substituents which can be cited are:
a linear or branched alkyl radical containing 1 to 6 carbon atoms, preferably 1 to 4 carbon atoms, such as methyl, ethyl, propyl, isopropyl, butyl, isobutyl, sec-butyl or tert-butyl;
a linear or branched alkoxy radical containing 1 to 6 carbon atoms, preferably 1 to 4 carbon atoms, such as methoxy, ethoxy, propoxy, isopropoxy, butoxy, isobutoxy sec-butoxy, tert-butoxy;
a hydroxy group;
a halogen atom, preferably a fluorine, chlorine or bromine atom.

Preferred acylation agents have formula (VIII) where X′ represents a chlorine atom and R₁₄ represents a methyl or ethyl radical.

When the acylation agent is an acid anhydride, the preferred compounds have formula (VIII) where R₁₄ and R₁₅ are identical and represent an alkyl radical containing 1 to 4 carbon atoms.

Regarding sulphonylation agents, they preferably have formula (IX) where X′ represents a chlorine atom or a —O—SO₂—R₁₅ is radical where R₁₅ represents an alkyl radical containing 1 to 4 carbon atoms and R₁₄ represents a phenyl or naphthyl radical or a R₁₆—O— radical or an (R₁₇)(R₁₈)—N— radical where R₁₆, R₁₇ and R₁₈ represent a linear or branched alkyl radical containing 1 to 4 carbon atoms.

Illustrative examples of acylation agents with formula (VIII) which can in particular be cited are:
acetyl chloride;
acetyl bromide;
monochloroacetyl chloride:
dichloroacetyl chloride;
propanoyl chloride;
isobutanoyl chloride;
pivaloyl chloride;
stearoyl chloride;
crotonyl chloride;
benzoyl chloride;
chlorobenzoyl chlorides;
p-nitrobenzoyl chloride;
o-nitrobenzoyl chloride;
methoxybenzoyl chlorides;
naphthoyl chlorides:
acetic anhydride;
isobutyric anhydride;
trifluoroacetic anhydride;
benzoic anhydride.

Examples of sulphonylation agents with general formula (IX) which can be mentioned include:
benzenesulphonyl chloride;
p-chlorobenzenesulphonyl chloride;
fluorobenzenesulphonyl chloride;
nitrobenzenesulphonyl chloride;
methoxybenzenesulphonyl chloride;
tosyl chloride;
dimethylsulphamoyl chloride;
methoxysulphonyl chloride;
benzenesulphonic anhydride;
p-toluenesulphonic anhydride.

Regarding the alkylation agent, a halide or alcohol can more particularly be used.

In the case of a halide type alkylation agent, the halogen atom can be chlorine, bromine or iodine, preferably one of the first two.

It can be symbolically represented by general formula (X):
R₁₈—X (X)

in which formula (X):
X represents a hydroxy group or a halogen atom; said hydroxy group or halogen atom must not be in the vinyl or alkynyl position;
R₁₈, which may be substituted, represents a linear or branched, saturated or unsaturated acyclic aliphatic radical; a saturated or unsaturated, monocyclic or polycyclic cycloaliphatic radical; or a linear or branched, saturated or unsaturated aliphatic radical carrying a cyclic substituent.

In formula (X), radical R₁₈ more particularly represents:
a linear or branched alkyl, alkenyl, alkadienyl or alkynyl radical, preferably containing 1 to 12 carbon atoms; the hydrocarbon chain can optionally be:
   interrupted by one of the following groups: 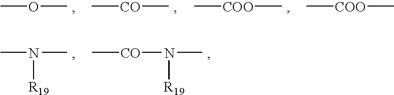
   in which formulae R₁₉ represents a hydrogen atom or an alkyl radical containing 1 to 4 carbon atoms, a cyclohexyl radical or a phenyl radical;
   and/or carrying one of the following substituents Y:
   an —OH group, a —COOH— group, a —CHO group, a —NO₂ group, a nitrile group, an amine group —NH₂, a halogen atom, preferably chlorine or bromine, or a —CF₃ group;
a cycloalkyl or cycloalkenyl radical preferably containing 5 to 7 carbon atoms, preferably carrying one or more substituents Y as defined above and/or one or more substituents Z which can be: a linear or branched alkyl radical containing 1 to 4 carbon atoms, a linear or branched alkoxy radical containing 1 to 4 carbon atoms, a radical with formula: 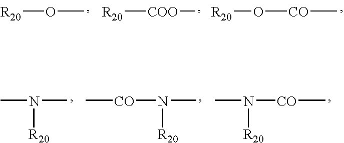
where R₂₀ has the meaning given above; said radicals can be bridged type polycycles;
a linear or branched, saturated or unsaturated acyclic aliphatic radical such as that defined above, carrying a cyclic substituent. The term “cycle” means a saturated, unsaturated or aromatic carbocylic or heterocyclic cycle.

The aliphatic acyclic radical can be connected to the cycle by a valence bond or by one of the following groups:

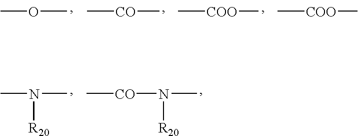

where R₂₀ has the meaning given above.

Examples of cycles are:
a saturated or unsaturated, monocyclic or polycyclic cycloaliphatic radical as mentioned above, preferably the cyclohexyl radical or the 1-cyclohexene-yl radical;
a phenyl, tolyl or xylyl radical, optionally carrying one or more Y or Z substituents;
a saturated, unsaturated or aromatic monocylic heterocyclic radical comprising one or more oxygen, nitrogen or sulphur atoms as the heteroatoms, containing 4 to 6 atoms in the cycle; said radical may carry one or more substituents Y or Z;
a radical constituted by a concatenation of 2 to 4 groups as defined above bonded together by a valence bond and/or by one of the following groups: 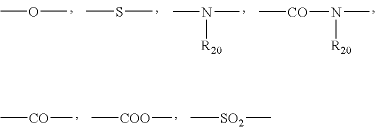
and/or by at least one alkoylene or alkoylidene group containing 1 to 4 carbon atoms; in said formulae, R₂₀ represents a hydrogen atom, an alkyl radical containing 1 to 4 carbon atoms, a cyclohexyl radical or a phenyl radical;
an aromatic radical constituted by a group of 2 to 3 aromatic carbocylic and/or heterocyclic cycles and forming orthocondensed systems between them, preferably benzene and pyridine nuclei.

Preferred examples of cyclo- or arylaliphatic radicals R₁₈ carrying a cyclic substituent which can be cited are the cyclohexylmethyl, cyclohexylbutyl, Benzyl, 2-phenylethyl, 2-[4-(2-butyl)phenyl]ethyl, styryl, α-phenylcyclohexylmethyl, phenoxymethyl and phenoxyethyl radicals.

In the definition of the radical R₁₈ for the halogenated derivative with formula (X), it should be noted that this carries one or more substituents Y or Z, the term “or more” generally means a total which does not exceed 3.

Preferably, radical R₁₈ represents:
a linear or branched alky radical containing 1 to 8 carbon atoms, such as the methyl, ethyl, propyl, isopropyl, butyl, isobutyl, sec-butyl, tert-butyl, 2-ethylbutyl, pentyl, isopentyl, neopentyl tert-pentyl, 2-methylpentyl, hexyl or 2-ethylhexyl radical,
a linear or branched alkenyl radical containing 2 to 8 carbon atoms and containing an ethylenic double bond β to the X group, for example the allyl, 3-methyl-2-butene-yl, or 3-hexene-yl radical;
a cyclohexyl radical which may be substituted by a linear or branched alkoyl radical containing 1 to 4 carbon atoms;
an arylalkyl radical as shown in the following formula: 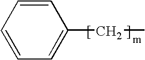
where m is a whole number in the range 1 to 4, preferably 1.

More particular examples of halide type alkylation agents with formula (X) which can be cited are:
methyl bromide;
ethyl bromide;
n-propyl bromide;
isopropyl bromide;
bromobutane;
bromohexane;
bromoheptane;
bromooctane;
1-bromo-2-methylbutane;
1-bromo-3-methylbutane;
2-bromo-2-methylbutane,
allyl bromide;
allyl chloride;
crotyl chloride;
3-chloro-2-methylpropene.
1-chloro-2-butene;
1-chloro-3-methyl-2-butene;
prenyl bromide;
geranyl bromide;
geranyl chloride;
α-bromoacetone;
α,α-dibromoacetone;
α-chloroacetone;
α,α′-chloroacetone;
methyl α-bromopropionate;
butyl α-bromopropionate;
methyl α-chloropropionate;
butyl α-chloropropionate;
cyclobutyl bromide;
cyclopentyl bromide;
cyclohexyl bromide;
cycloheptyl bromide;
(bromomethyl)cyclopropane;
(2-chloromethyl)pyridine;
(3-chloromethyl)pyridine;
(4-chloromethyl)pyridine;
benzyl bromide;
benzyl chloride;
(4-chloromethyl)stilbene;
(1-bromomethyl)naphthalene:
(2-bromomethyl)naphthalene.

Of the halides cited above, the following are preferred:
allyl bromide:
allyl chloride;
benzyl bromide;
benzyl chloride;
isopropyl bromide;
crotyl chloride;
1-chloro-2-butene;
3-chloro-2-methylpropene;
cyclohexyl bromide.

Regarding the choice of halide used, either chloride or bromide, it is determined as function of the nature of the hydrocarbon from which the halide derives and of economic strictures.

In carrying out the process of the invention, a brominated compound is used in all cases.

However, as chlorides are cheaper, they should be used preferentially but they cannot always be used. A chloride type halide cannot be used in the process of the invention if there is an unsaturated bond in the hydrocarbon chain carrying the chlorine atom in the position α to the latter: the unsaturated bond could be a double or triple bond. As an example, benzyl chloride can advantageously be used.

Examples of alcohol type alkylation agents which can in particular be cited are:
benzyl alcohol;
β-phenylethyl alcohol.

The acylation, sulphonylation or alkylation reactions are carried out conventionally in the presence of a Friedel-Crafts type catalyst.

The catalyst used in the process of the invention is a Friedel-Crafts type catalyst.

A first class of catalysts suitable for the invention is constituted by Lewis acids.

Examples of organic salts which can be cited are the acetate, propionate, benzoate, methanesulphonate and trifluoromethanesulphonate of metallic elements or metalloids from groups (IIIa), (IVa), (VIII), (IIb), (IIIb), (IVb), (Vb) and (VIb) of the periodic table.

Regarding inorganic salts, the chloride, bromide, iodide, sulphate, oxide and analogous products of metallic elements or metalloids from groups (IVa), (VIII), (IIb) (IIIb), (IVb), (Vb) and VIb) of the periodic table can be used.

In the present text, reference shall be made to the periodic table published in the Bulletin de la Société Chimique de France, no 1 (1966).

The salts used in the process of the invention are more particularly those from elements from group (IIIa) of the periodic table, preferably scandium, yttrium and the lanthanides; from group (IVa), preferably titanium, zirconium; from group (VIII), preferably iron; from group (IIb), preferably zinc; from group (IIIb), preferably boron, aluminium, gallium, indium; from group (IVb), preferably tin; from group (Vb), preferably bismuth; from group (VIb), preferably tellurium.

Of the inorganic salts, metallic halides can be cited, preferably zirconium chloride, ferric chloride, zinc chloride, aluminium chloride, aluminium bromide, gallium chloride, indium chloride, stannic chloride, bismuth chloride, boron trifluoride; ferrous oxide, ferric oxide, and gallium oxide.

A halide can be generated in situ using a known method (International patent application PCT/FR98/00497, publication number 98/40339).

Preferred examples of catalysts which can be cited are aluminium chloride, boron trifluoride, titanium tetrachloride and tin tetrachloride.

Regarding organic salts, rare earth and/or bismuth salts of trifluoromethanesulphonic acid (commonly known as triflic acid) are preferably used.

The term “rare earth” means lanthanides with an atomic number or 57 to 71, also yttrium and scandium.

The process of the invention more particularly envisages using the following rare earths: lanthanum, ytterbium, lutetium and/or scandium.

Rare earth triflates are known products which have been described in the literature, in particular in U.S. Pat. No. 3,615,169. They are generally obtained by reacting a rare earth oxide with trifluoromethanesulphonic acid.

Bismuth salts of triflic acid described in International patent application PCT/FR96/01488 can also be used in the process of the invention.

A further class of catalysts which is suitable for the invention is constituted by Brönsted acids, in particular sulphuric acid, hydrofluoric acid, hydrochloric acid, phosphoric acids and polyphosphoric acids sulphonic acids and in particular trifluoromethanesulphonic acid, perfluorosulphonic acid and fluorosulphonic acid,

In the process of the invention, a solid catalyst as defined above is used which may also be supported. To this end, the support can be selected from metal oxides such as aluminium oxides, silicon and/or zirconium oxides, clays, more particularly kaolin, talc or montmorillonite, or from charcoal, possibly activated by a known treatment with nitric acid, acetylene black or resins.

The support can be in any form, for example a powder, beads, granules, extrudates.

In the catalyst, the amount of active phase represents 5% to 100% of the weight of the catalyst.

In accordance with the process of the invention, the reaction between the phenolic compound and the acylation, sulphonylation or alkylation agent is carried out in the liquid phase, in the presence or absence of an organic solvent; one of the reactants can be used as the reaction solvent.

In a preferred variation of the process of the invention, the reaction is carried out in an organic solvent.

A number of factors govern the choice of solvent.

It must be inert under the conditions of the invention, and must have a boiling point which is higher than the reaction temperature.

Preferably, an aprotic, low polarity organic solvent is used.

Examples of solvents which are suitable for the present invention which can in particular be cited are aliphatic or aromatic hydrocarbons, which may or may not be halogenated.

Examples of aliphatic hydrocarbons which can in particular be cited are paraffins such as hexane, heptane or cyclohexane and aromatic hydrocarbons, in particular aromatic hydrocarbons such as benzene, toluene, xylenes, cumene, and petroleum cuts constituted by a mixture of alkylbenzenes in particular Solvesso® type cuts.

Regarding aliphatic or aromatic halogenated hydrocarbons, the following can in particular be cited: perchlorinated hydrocarbons such as tetrachloromethane: partially chlorinated hydrocarbons such as dichloromethane, chloroform, 1,2-dichloroethane and trichloroethylene; and halogenated aromatic hydrocarbons such as monochlorobenzene, dichlorobenzenes and mixtures thereof.

It is also possible to use mixture of organic solvents.

The electrophilic substitution reaction is carried out using the reactants in the proportions mentioned below.

The ratio between the number of moles of phenolic compound and the number of moles of acylation, sulphonylation or alkylation agent can vary as the substrate may act as the reaction solvent. Thus the ratio can be from 0.1 to 10, preferably about 4.0.

The quantity of catalyst used is determined such that the ratio between the number of moles of catalyst and the number of moles of acylation, sulphonylation or alkylation agent is preferably in the range 0.001 to 1.0, more preferably in the range 0.02 to 0.2.

Regarding the quantity of organic solvent used, it is generally selected such that the ratio between the number of moles of organic solvent and the number of moles of phenolic compound is preferably in the range 0 to 100, more preferably in the range to 50.

The temperature at which the Friedel-Crafts reaction is carried out depends on the reactivity of the starting substrate and that of the acylation agent.

It is in the range 20° C. to 200° C., preferably in the range 40° C. to 150° C.

In general, the reaction is carried out at atmospheric pressure but lower or higher pressures can also be suitable.

From a practical viewpoint, there are no restrictions on how the reactants are processed. They can be introduced in any order.

After bringing the reactants into contact, the reaction mixture is brought to the desired temperature.

A further variation of the invention consists of heating one of the reactants (acylation, sulphonylation or alkylation agent or phenolic compound) with the catalyst then introducing the other reactant.

The reaction duration depends on a number of parameters. It is usually 30 minutes to 8 hours.

At the end of the reaction, the reaction medium is treated conventionally to eliminate the catalyst, in particular using a water hydrolysis treatment.

The acylated, sulphonated or alkylated protected phenolic compound is recovered from the organic phase using known techniques, by eliminating the organic solvent by distillation or crystallisation.

The hydroxy group de-protection step can be carried out preferably by using the mode carried out in the basic medium described above.

Aminoalkylation

In accordance with the process of the invention, a protected phenolic compound with formula (II) is condensed with a carbonyl compound with formula (XI) in the presence of a secondary amine.

A carbonyl compound with general formula (XI) is involved:

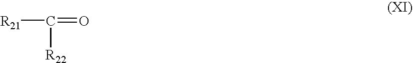

in which formula (XI):
R₂₁ represents a hydrogen atom, a linear or branched alkyl radical containing 1 to 6 carbon atoms, an alkenyl radical containing 2 to 6 carbon atoms, or a phenyl radical which may be substituted;
R₂₂ can be equal to R₂₁ or it can represent an electron-attracting group.

Examples of electron-attracting groups which are suitable for the present invention which can be cited are:
a —CHO group;
a R₂₃—CO— or R₂₃—CO—(CH₂)ₘ— acyl group where R₂₃ represents a linear or branched alkyl radical containing 1 to 6 carbon atoms and m represents a number from 1 to 3;
a —COOR₂₄ group where R₂₄ represents a hydrogen atom or a linear or branched alkyl radical containing 1 to 6 carbon atoms;
a —CX₂H group where X represents a halogen atom, preferably a fluorine, chlorine or bromine atom;
a —CX₃ group where X represents a halogen atom, preferably a fluorine, chlorine or bromine atom.

Examples of carbonyl compounds with formula (XI) which can be cited are:
formaldehyde or a formaldehyde generator such as paraformaldehyde which originates from an aqueous solution (methylene glycol) or used in the form of linear polyformaldehydes with any degree of polymerisation, preferably with a number of (CH₂O) motifs in the range 8 to 100 motifs;
glyoxal;
ethanal;
propanal;
benzaldehyde
glyoxylic acid;
chloral;
bromal;
fluoral;
acetone;
methylethylketone;
methylisobutylketone;
dichloroacetone;
trifluoroacetone;
trifluoroacetophenone;
trifluoroacetylacetone;
methyl pyruvate;
ethyl pyruvate.

Paraformaldehyde is preferred.

Said reactant is generally used in the form of an aqueous solution with a concentration of less than 50% by weight, preferably in the range 20% to 50% by weight. It can contain a few percent of alcohol, generally methanol in an amount of less than 15% by weight,

Examples of amines which are suitable for the invention which can be cited are those with formula (XII) below:

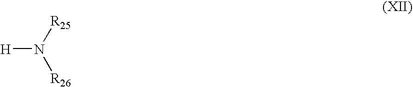

where:
R₂₅ and R₂₆, which may be identical or different, represent:
   a linear or branched alkyl radical containing 1 to 12 carbon atoms, a secondary alkyl radical containing 3 to 12 carbon atoms or a tertiary alkyl radical containing 4 to 12 carbon atoms; the alkyl radicals may contain one or two ether functions —O— or hydroxy, tertiary amine, carboxylic acid or carboxylic acid ester functions,
   a phenyl radical, a cyclohexyl radical, a cycloheptyl radical or a cyclopentyl radical;
   a phenylalkyl, cyclohexylalkyl, cycloheptylalkyl or cyclopentylalkyl radical wherein the alkyl portion contains 1 to 4 carbon atoms;
   a hydrogen atom;
R₂₅ or R₂₆, together with the nitrogen atom form a heterocycle which may be saturated or contain one or more double bonds, optionally substituted with one or more alkyl radicals containing 1 to 4 carbon atoms;
R₂₅ and R₂₆ together with the nitrogen atom and with one or more nitrogen and/or oxygen and/or sulphur atoms form a saturated or unsaturated heterocycle optionally substituted by one or more alkyl radicals containing 1 to 4 carbon atoms;
R₂₅ and R₂₆ together with the nitrogen atom form an unsaturated heterocycle optionally substituted by one or two methyl or ethyl groups;
R₂₅ and R₂₆ together with the nitrogen atom and optionally with one or more nitrogen and/or oxygen and/or sulphur atoms form a saturated or unsaturated polycyclic compound optionally substituted by one or more alkyl radicals containing 1 to 4 carbon atoms.

For the purposes of the present invention, preferred amines with general formula (XII) are secondary amines with formula (XIIa):

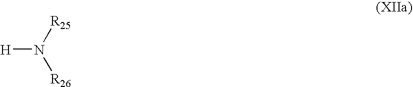

where:
R₂₅ and R₂₆, which may be identical or different, represent:
   a linear alkyl radical containing 1 to 10 carbon atoms;
   a secondary alkyl radical containing 3 to 10 carbon atoms;
   a tertiary alkyl radical containing 4 to 10 carbon atoms;
   a cyclohexyl or cyclopentyl radical;
   a phenyl radical;
   a benzyl or phenethyl radical;
R₂₅ and R₂₆ together with the nitrogen atom and with a further nitrogen and/or oxygen atom form a heterocycle which is such saturated or comprises one or more unsaturations:
R₂₅ and R₂₆ comprise one or more amine, hydroxy or carboxylic acid ester functions.

Illustrative examples which can be cited as amines with formula (XII) or (XIIa) are dimethylamine, diethylamine, dipropylamine, diisopropylamine, dibutylamine, diisobutylamine, methylpropylamine, methylbutylamine, methylisobutylamine, methyltertiobutylamine, dilaurylamine, methylbenzylamine, ditertiobutylamine, 1-methylcyclopentylamine, 1-methylcyclohexylamine, dicyclohexylamine, morpholine, imidazole, pyrrolidine, imidazolidine and piperazine.

Preferably, dimethylamine, diethylamine, dipropylamine, diisopropylamine, dibutylamine or dilaurylamine are used.

Preferred conditions for the concentrations and quantities of reactants to be used will be given below.

In the process of the invention, the carbonyl compound with formula (XI) is reacted with the protected phenolic compound with formula (II) in the presence of a secondary amine.

Preferably, the quantity of protected phenolic compound, is in excess with respect to the secondary amine.

The ratio between the number of moles of protected phenolic compound with formula (II) and the number of moles of secondary amine with formula (XII) is in the range 0.9 to 3.0, preferably in the range 1.0 to 2.0.

The quantity of secondary amine is generally in excess with respect to the carbonyl compound. The ratio between the number of moles of secondary amine and the number of moles of carbonyl compound is advantageously in the range 1.0 to 1.2.

In accordance with the process of the invention, the reaction is preferably carried out in an organic solvent, preferably a low polarity aprotic solvent.

Examples of solvents which are suitable for use in the present invention which can in particular be cited are aliphatic or aromatic hydrocarbons, which may or may not be halogenated and aliphatic, cycloaliphatic or aromatic ether-oxides.

The organic solvents can also be aliphatic, cycloaliphatic or aromatic ether-oxides, more particularly diisopropyl oxide, methyltertiobutylether, ethylene elycol dimethylether (or 1,2-dimethoxyethane), diethylene glycol dimethylether (or 1,5-dimethoxy-3-oxapentane), biphenyl or benzyl oxide, dioxane or tetrahydrofuran (THF).

Preferred solvents are: aromatic hydrocarbons, more particularly benzene and toluene.

It is also possible to use a mixture of solvents.

The concentration of protected phenolic compounds in the solvent can vary within wide limits. Thus generally, it is also possible to use 0.25 to 2 litres, preferably 0.5 to 1 litre of solvent per mole of protected phenolic compound.

In a variation of the process of the invention, a catalyst is added which facilitates the reaction between the secondary amine and the carbonyl compound, and the reaction of the resulting product with the protected phenolic compound.

Examples of catalysts which can be used include sulphuric acid, halogenated or non halogenated sulphonic acids, more particularly halogenosulphonic acids such as fluorosulphonic acid, chlorosulphonic acid or trifluoromethanesulphonic acid, methanesulphonic acid, ethanesulphonic acid, ethanedisulphonic acid, camphorsulphonic acid, benzenesulphonic acid, benzenedisulphonic acids, toluenesulphonic acids, naphthalenesulphonic acids and naphthalenedisulphonic acids.

The quantity of catalyst, expressed in moles of catalyst per mole of protected phenolic compound with formula (II), can vary within wide limits. It is generally of the order of 1%.

The temperature at which the reaction is carried out is advantageously in the range 60° C. to 120° C.

The reaction is preferably carried out at atmospheric pressure.

When using an organic solvent with too low a boiling point, the process of the invention can be carried out under pressure.

In the opposite case where the boiling point of the solvent is too high, the process of the invention can be carried out under reduced pressure, advantageously in the range 100 mm (13300 Pa) to 500 mm (46500 Pa) of mercury.

In a preferred variation of the process of the invention, the process of the invention is carried out under a controlled atmosphere of inert gases. An atmosphere of rare gases can be established, preferably argon, but nitrogen is more economical.

From a practical viewpoint, the process of the invention is simple to carry out.

The different reactants can be introduced in any order. Preferably, the following order is used: the carbonyl compound is introduced then the secondary amine to form a hemiaminal reactant, then the protected phenolic compound and the acid catalyst.

The reaction medium is brought to the desired temperature, stirring the reaction medium at the temperature desired in the range given above for a period of 1 to 10 hours.

Water forms in the reaction medium during the reaction. In a preferred variation of the invention, it is eliminated from the reaction mixture as it is formed by any known means, in particular by azeotropic distillation.

At the end of the reaction, a protected phenolic compound carrying an aminoalkyl group in the position para to the electron-donating group is obtained.

The hydroxy group de-protection step is preferably carried out by conducting the process in a basic medium as described above.

Hydroxyalkylation

In a second step the process of the invention employing a hydroxyalkylation reaction.

To this end, the protected phenolic compound, preferably with formula (II), is reacted with a carbonyl compound in the presence of a suitable catalyst.

More particularly, the carbonyl compound which can be used has general formula (XI) described above.

More specific examples of carbonyl compounds have also been given.

Of the carbonyl compounds cited above, formaldehyde is preferred.

Said reactant is generally used in the form of an aqueous solution with a concentration of less than 50% by weight, preferably in the range 20% to 50% by weight.

The hydroxyalkylation reaction is carried out in the presence of a catalyst which is usually used for such a reaction.

A homogeneous catalyst can be used, in particular one of the Brönsted acids cited above.

It is also possible to use a heterogeneous catalyst and more particularly zeolites, in particular those described in the literature, more particularly in EP-A-0 773 918.

Preferred zeolites for use in the process of the invention are β zeolites and mordenites.

The zeolites used in the process of the invention are known products which have been described in the literature [see the “Atlas of Zeolite Structure Types” by W. M. Meier and D. H. Olson, published by the Structure Commission of the International Zeolite Association (1992)].

It may be necessary to carry out a conventional zeolite dealumination treatment or to carry out a treatment rendering it acidic in particular using an acid such as hydrochloric acid, sulphuric acid, nitric acid, perchloric acid, phosphoric acid and trifluoromethanesulphonic acid.

The zeolite constitutes the catalytic phase. It can be used alone or mixed with a mineral matrix. The matrix can be selected from metal oxides such as aluminium, silicon and/or zirconium oxides, or from clays, in particular kaolin, talc or montmorillonite.

The amount of active phase in the catalyst represents 5% to 100% of the catalyst weight The catalysts can be in different forms in the process of the invention: powder, formed products such as granules (for example extrudates or beads), pellets, which are obtained by extrusion, moulding, compacting or any type of known process.

The protected phenolic compound with formula (II) is preferably reacted with the carbonyl compound with formula (XI) in an aqueous medium when formaldehyde is used. However, it is also possible to carry out the reaction in an organic liquid which is inert under the selected reaction conditions.

Examples of suitable solvents which can be cited are aliphatic or aromatic hydrocarbons, which may or may not be halogenated, and aliphatic, cycloaliphatic or aromatic ether-oxides.

Polar aprotic solvents can be used, such as nitro compounds, for example nitrobenzene: aliphatic or aromatic nitriles such as acetonitrile: or tetramethylenesulphone (sulpholane).

The concentration of protected phenolic compound in the medium can vary within wide limits. It can be in the range 0.1 to 5 moles per litre of medium, preferably in the range 0.2 to 2 moles per litre.

The catalyst can represent 5% to 80%, preferably 10% to 50% by weight with respect to the weight of protected phenolic compound used.

The quantity of carbonyl compound with formula (XI) expressed in moles of carbonyl compound per mole of protected phenolic compound with formula (II) can also vary within wide limits. The mole ratio of the carbonyl compound with formula (XI)/protected phenolic compound with formula (II) can be in the range 1 to 50. The upper limit is not critical in nature but for economical reasons, it should not be exceeded.

The temperature of the hydroxyalkylation reaction is advantageously in the range 20° C. to 100° C. more preferably in the range 40° C. to 90° C.

Generally, the reaction is carried out at atmospheric pressure but higher pressures of 1 to 50 bars may be suitable, preferably 1 to 25 bars. Autogenous pressure conditions are employed when the reaction temperature is higher than the boiling point of the reactants and/or products.

The reaction is preferably carried out under a controlled atmosphere of inert gas such as nitrogen or a rare gas, for example argon.

The duration of the reaction is highly variable. It is usually in the range 15 minutes to 10 hours, preferably in the range 30 minutes to 5 hours.

From a practical viewpoints the process can be carried out batchwise or continuously.

The catalyst, carbonyl compound with formula (XI), and optional organic solvent can be charged then the protected phenolic compound can be introduced. In a preferred implementation of the invention, the protected phenolic compound is gradually introduced continuously or in fractions, then the reaction mixture is brought to the desired temperature.

At the end of the reaction, a liquid phase is recovered, comprising the protected phenolic compound hydroxyalkylated in the position para to the electron-donating group which can be recovered conventionally.

In a variation of the invention, the protected phenolic compound carrying a hydroxyalkylated group can be oxidised to a formyl group using a process consisting of liquid phase oxidation using molecular oxygen or a gas containing molecular oxygen in the presence of a catalyst based on a metal M selected from metals from group 8 of the periodic table, optionally comprising, as activators for the metals, metals such as cadmium, cerium, bismuth, lead, silver, tellurium or tin.

Reference should be made in this regard to the literature, in particular to EP-A-0 773 918.

The hydroxy group de-protection step is preferably carried out employing the implementation involving a basic medium as described above.

Formylation

A further reaction consists in reacting the protected phenolic compound which preferably has formula (II) with chloral or hexamethylenetetramine.

Said reactants are used in a quantity, expressed with respect to the phenolic compound, of at least the stoichiometric quantity and usually in excess, for example up to 200%.

The reaction can be carried out in bulk in an acid such as trifluoroacetic acid, sulphuric acid, hydrofluoric acid or boric acid.

Generally, a quantity of acid equal to twice the stoichiometric quantity, expressed with respect to the protected phenolic compound, is used.

The reaction is advantageously carried out between 60° C. and 100° C.

The protected phenolic compound with a formyl group para to the electron-donating group is recovered.

The hydroxy group de-protection step is preferably carried out using the basic medium implementation described above.

The invention is applicable to all electrophilic substitution reactions which can introduce a C—C or C—S bond.

It is not limited to the reactions mentioned above which constitute examples only of the electrophilic substitution reaction.

Examples of the process of the invention will now be given.

### EXAMPLE 1

The following compound (A) was prepared:

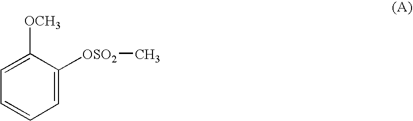

50 g of guaiacol and 100 ml of ether were placed in a twin-necked flask stirred with a magnetic stirrer and provided with a dropping funnel.

46.14 g of methanesulphonyl chloride was added using a syringe, with stirring.

56 ml of triethylamine diluted in 20 ml of ether was added over 3 hours.

The triethylamine salts which precipitated out were filtered.

The mother liquors were recovered and concentrated.

Mesylated gualacol was observed to precipitate out.

Two re-crystallisation steps were carried out from ether and cold crystallised (−20° C.).

76.27 g of a white product with a melting point of 35-37° C. was recovered.

### EXAMPLE 2

The following compound (B) was prepared:

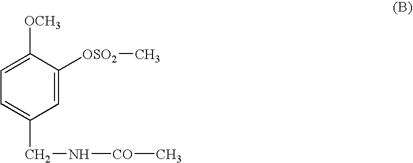

the following were charged into a reactor:
8.19 g of acetamide;
2.2 g of paraformaldehyde;
9 ml of acetic acid;
6 ml of sulphuric acid.

When the solution was clear, i.e., all of the paraformaldehyde had dissolved, mesylated guaiacol (2-methylsulphone-1-methoxyphenol) was added.

It was heated for 15 hours at the temperature of the oil bath, namely 80-90° C.

The medium was allowed to cool to ambient temperature then it was diluted with ethyl acetate (50 ml).

An aqueous 2N potassium hydroxide solution was added until the pH was about 8.

The product formed was extracted twice in succession (2×50 ml) with ethyl acetate and once (50 ml) with trichloromethane.

The organic fractions were combined.

They were dried over magnesium sulphate.

The product obtained was concentrated in a rotary evaporator.

The product was crystallised from ethyl acetate (or trichloromethane).

Product (B) was obtained in a yield, expressed with respect to the mesylated guaiacol, of 80%.

### EXAMPLE 3

In this example, compound (C) was prepared; followed by de-protection of compound (B) obtained in Example 2.

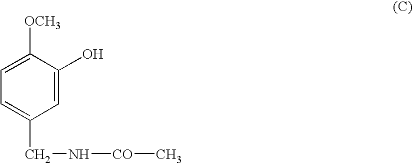

4 g of the starting product (B) was dissolved in 20 ml of degassed isopropanol.

40 ml of a 5% by weight aqueous potassium solution, also degassed, was added.

It was heated under reflux for 6 hours, the temperature of the bath being 100-110° C.

The crude reaction mixture obtained was concentrated in a rotary evaporator.

The solid obtained was washed using 50 ml of trichloromethane.

5 g of potassium carbonate was added and it was allowed to rotate for about 1 hour.

The crude product obtained was filtered through a paper filter.

The filtrate was concentrated using a rotary evaporator and solvent traces were eliminated under reduced pressure (10 mm of mercury).

2.5713 g of a yellow-white solid (C) was recovered, corresponding to a yield of 90%.

The structure of product (C) obtained was confirmed by NMR using CDCl.

### EXAMPLE 4

A compound (D) was prepared by acid treatment of compound (B).

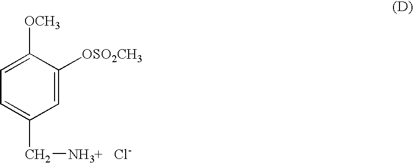

An aqueous solution of 2 N hydrochloric acid was prepared and degassed.

The starting product was dissolved in 50 ml of this hydrochloric acid solution.

It was heated for 6 hours at the oil bath temperature (110° C.).

The crude reaction mixture was concentrated in a rotary evaporator.

The structure was confirmed by CDCl₃ NMR.

### EXAMPLE 5

The following formylation reaction was carried out:

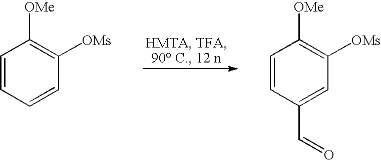

(Ms=OSO₂CH₃).

Mesylated guaiacol (0.5 g, 2.5 mmol), hexamethylenetetramine (0.35 g (2.5 mmol) and trifluoroacetic acid (3 ml) were introduced in succession into a flask.

The mixture was heated under reflux for 12 hours, then brought to ambient temperature.

20 ml of ice water was then added.

After stirring for 15 minutes, the pH was adjusted to between 8 and 9 by adding a saturated sodium carbonate solution.

The aqueous phase was then extracted with ether (3×50 ml).

The organic phases were combined, dried over sodium sulphate then concentrated under reduced pressure (10 mm of mercury).

The product was obtained in a yield of 90% after purification by silica gel chromatography using a 5/5 ethyl acetate/hexane mixture as the eluent.

¹H NMR (300 MHz, CDCl₃), δ (ppm): 3.25 (3H.s), 4.00 (3H.s, 7.17 (1H, d. J=8.3 Hz), 7.87 (2H, d, 8.3 Hz), 9.8 (1H, s).

### EXAMPLE 6

The following Friedel-Crafts acylation reaction was carried out:

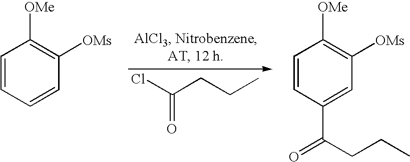

A mixture of butyryl chloride (0.42 g 4 mmol) and mesylated guaiacol (0.8 g, 4 mmol) was added dropwise at −5° C. over 40 min to aluminium chloride (0.64 g, 4.8 mmol) in solution in nitrobenzene (6 ml).

The temperature was allowed to return to ambient temperature (AT) and the reaction mixture was stirred for 12 hours.

After this time, 25 ml of water was added then the pH was adjusted to between 8 and 9 using a saturated sodium carbonate solution.

The aqueous phase was extracted three times with ethyl acetate (50 ml).

The organic phases were combined, dried over sodium sulphate then concentrated under reduced pressure (10 mm of mercury).

The product was obtained in a yield of 65% after purification by silica gel chromatography using an 8/2 ethyl acetate/hexane mixture as the eluent.

¹H NMR (300 MHz, CDCl₃), δ (ppm): 1.04 (3H, t, J=7.3 Hz), 1.78 (2H, m), 2.9 (2H, t, J=7.3 Hz), 3.24 (3H, s), 3.98 (3H, s), 7.08 (1H, d, J=8.3 Hz), 7.93 (2H, m).

1-52. (canceled)

53. A process for functionalisation in the position para with respect to an electron-donating group carried by a starting phenolic compound, said process comprising the following steps:
a) protecting the hydroxy group of the phenolic compound by means of a sulphonic ester function;
b) reacting the protected phenolic compound of step a) with an electrophilic reactant which comprises at least one electrophilic carbon atom or an electrophilic sulphur atom in order to introduce a C—C or C—S bond; and
c) recovering the product obtained in step b).

54. A process according to claim 53, further comprising deprotecting the hydroxy group of the phenolic compound before step c).

55. A process according to claim 53, wherein the starting phenolic compound carries at least one electron-donating group in the position ortho to the hydroxy group and a hydrogen atom in the position para to the electron-donating group.

56. A process according to claim 53, wherein the starting phenolic compound has general formula (I): 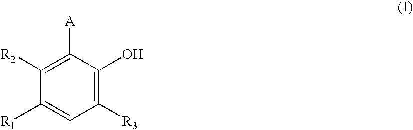
wherein:
A represents an electron-donating group;
R₁, R₂ and R₃, which are identical or different, represent:
a hydrogen atom;
a linear or branched alkyl radical containing 1 to 20 carbon atoms, optionally carrying one or more halogen atoms;
a cycloalkyl group containing 3 to 8 carbon atoms;
an aralkyl radical containing 6 to 20 carbon atoms;
an aryl radical containing 6 to 20 carbon atoms;
a halogen atom; or
an electron-attracting group.

57. A process according to claim 56, wherein two groups R₁ and R₂ together with the two atoms carrying them, form a saturated, unsaturated or aromatic carbocyclic cycle containing 3 to 8 carbon atoms.

58. A process according to claim 56, wherein the electron-attracting group is a carboxy group, an ester group, a nitrile or a nitro group.

59. A process according to claim 56, wherein R₁, R₂ and R₃ represent a hydrogen atom, a linear alkyl group containing 1 to 4 carbon atoms, a branched alkyl group containing 1 to 4 carbon atoms, a halogen atom or a trifluoromethyl group.

60. A process according to claim 56, wherein A represents:
a linear or branched alkyl group containing 1 to 6 carbon atoms;
a cycloalkyl group containing 3 to 8 carbon atoms;
a phenyl radical;
a benzyl radical;
a phenylethyl radical;
a hydroxy group;
a fluorine atom;
an alkoxy group containing 1 to 6 carbon atoms in the alkyl portion;
a phenoxy group;
a di-substituted amino group, wherein the substituents, which are identical or different, are linear or branched alkyl radicals containing 1 to 6 carbon atoms, or a phenyl group;
an alkylamide wherein the alkyl group contains 1 to 6 carbon atoms; or
an arylamide wherein the aryl group contains 6 to 12 carbon atoms.

61. A process according to claim 60, wherein A represents a methoxy or an ethoxy group.

62. A process according to claim 56, wherein in step a), the hydroxy function is transformed into a sulphonic ester group.

63. A process according to claim 62, wherein said sulphonic ester group is:
tosylate (p-toluenesulphonate) —OSO₂—C₆H₄—CH₃;
brosylate (p-bromobenzenesulphonate) —OSO₂—C₆H₄—Br;
nosylate (p-nitrobenzenesulphonate) —OSO₂—C₆H₄—NO₂;
mesylate (methanesulphonate) —OSO₂—CH₃;
betylate (ammonioalkanesulphonate) —OSO₂—(CH₂)ₙNMe₃⁺ wherein n is in the range 0 to 6;
triflate (trifluoromethanesulphonate) —OSO₂—CF₃;
nonaflate (nonafluorobutanesulphonate) —SO₂—C₄F₉; or
tresylate (2,2,2-trifluoroethanesulphonate) —SO₂—CH₂—CF₃.

64. A process according to claim 53, wherein the protected form of the phenolic compound has general formula (II): 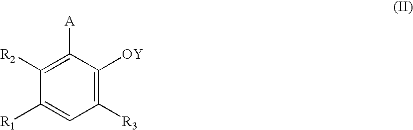
wherein:
Y represents the following group: 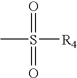
wherein R₄ represents a hydrocarbon radical containing 1 to 20 carbon atoms, and A, R₁, R₂ and R₃ have the meanings given above.

65. A process according to claim 64, wherein Y is:
an alkyl radical containing 1 to 10 carbon atoms, optionally carrying a halogen atom, a CF₃ group or an ammonium group N(R₅)₄, wherein the R₅ groups, which are identical or different, represent an alkyl radical containing 1 to 4 carbon atoms;
a cycloalkyl radical containing 3 to 8 carbon atoms;
a phenyl radical optionally carrying an alkyl radical containing 1 to 10 carbon atoms, a halogen atom, a CF₃ group or a NO₂ group;
a CX₃ group where X represents a fluorine, chlorine or bromine atom; or
a CF₂—CF₃ group.

66. A process according to claim 64, wherein the protected phenol with formula (II) is obtained by reacting, in the presence of a base, a starting phenolic compound with formula (I) with a protecting agent with the following formula (III): 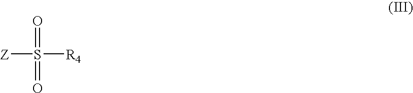
wherein:
R₄ has the meaning given above; and
Z represents a hydroxy group; a halogen atom; a —O—SO₂—R₄′ group, wherein R₄′, which is identical to or different from R₄, has the meaning given for R₄.

67. A process according to claim 66, wherein Z represents a chlorine or a bromine atom.

68. A process according to claim 53, wherein the protecting agent is triflic anhydride; methanesulphonyl chloride; benzenesulphonyl chloride; or p-toluenesulphonyl chloride.

69. A process according to claim 62, further comprising the use of a mineral or organic base.

70. A process according to claim 69, wherein said base is an alkali metal hydroxide; an alkali metal carbonate, an alkali metal bicarbonate, an alkali metal phosphate, an alkali metal hydrogen phosphate, an alkali metal acetate, an alkali metal trifluoroacetate; or a tertiary amine.

71. A process according to claim 70, wherein the tertiary amine has general formula (IV):
N—(R₆)₃ (IV)
wherein the groups R₆, which are identical or different, represent hydrocarbon residues containing 1 to 20 carbon atoms, optionally two R₆ groups together with the nitrogen atom, form a heterocycle containing 4 to 5 atoms.

72. A process according to claim 71, wherein the amine is triethylamine, tri-n-propylamine, tri-n-butylamine, methyldibutylamine, methyldicyclohexylamine, ethyldiisopropylamine, N,N-diethylcyclohexylamine, dimethylamino-4-pyridine, N-methylpiperidine, N-ethylpiperidine, N-n-butylpiperidine, 1,2-dimethylpiperidine, N-methylpyrrolidone, or 1,2-dimethylpyrrolidine.

73. A process according to claim 62, wherein the phenolic compound protection reaction is carried out in an organic, polar or apolar solvent, which is an aliphatic hydrocarbon optionally halogenated, a cycloaliphatic hydrocarbon optionally halogenated; an ester; an ether or a nitrile.

74. A process according to claim 69, wherein the quantity of base, expressed as the ratio of the number of equivalents of OH⁻ to the number of moles of phenolic compound with formula (I), is in the range 0.5 to 3.0.

75. A process according to claim 62, wherein the quantity of protecting agent, expressed as the ratio between the number of moles of protecting agent and the number of moles of starting phenolic compound, is in the range 1.0 to 1.5.

76. A process according to claim 62, wherein the temperature of step a) at which the operation for protecting the hydroxy group of the phenolic compound is carried out, is between 0° C. and ambient temperature.

77. A process according to claim 62, wherein before step b), the protected the phenolic compound is further purified by re-crystallisation from an organic solvent.

78. A process according to claim 53, wherein in step b) the protected phenolic compound is reacted with an electrophilic reactant in an amidoalkylation reaction, a Friedel-Crafts reaction, an aminoalkylation reaction, a hydroxyalkylation reaction, a formylation reaction, a carboxylation reaction or a further electrophilic substitution reaction.

79. A process according to claim 78, wherein the protected phenolic compound, is reacted, in the presence of a strong acid, with an amide or a carbamate and a carbonyl compound with general formula (V): 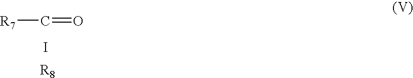
wherein:
R₇ represents a hydrogen atom; and
R₈ represents a hydrogen atom, a linear or branched alkyl radical containing 1 to 6 carbon atoms, a cycloalkyl radical containing 3 to 8 carbon atoms, an alkenyl radical containing 2 to 6 carbon atoms, a phenyl radical optionally substituted, or an electron-attracting group.

80. A process according to claim 79, wherein the electron-attracting group is:
a —CHO group;
a R₉—CO— or R₉—CO—(CH₂)ₘ— group wherein R₉ represents a linear or branched alkyl radical containing 1 to 6 carbon atoms and m represents a number from 1 to 3;
a —COOR₁₀ group wherein R₁₀ represents a hydrogen atom or a linear or branched alkyl radical containing 1 to 6 carbon atoms;
a —CX₂H group wherein X represents a halogen atom;
a —CX₃ group wherein X represents a halogen atom; or
a nitro group.

81. A process according to claim 79, wherein the carbonyl compound with formula (V) is formaldehyde, a formaldehyde generator; glyoxal, glyoxylic acid, bromal or fluoral.

82. A process according to claim 79, wherein the amide or carbamate used has the following formula (VI): 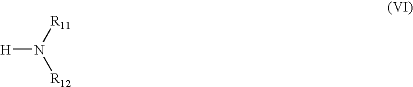
wherein:
at least one of groups R₁₁ and R₁₂ represents a group: 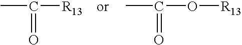
wherein the groups R₁₁, R₁₂ and R₁₃, which are identical or different, represent:
a hydrogen atom;
a linear or branched alkyl radical containing 1 to 12 carbon atoms, optionally carrying halogen atoms;
a cycloalkyl radical containing 3 to 8 carbon atoms;
a cycloalkylalkyl radical wherein the alkyl portion contains 1 to 4 carbon atoms;
an alkylaryl radical, wherein the alkyl portion contains 1 to 4 carbon atoms; and, optionally
two groups R₁₁ and R₁₂, together with the two atoms carrying them, further form a saturated, unsaturated or aromatic carbocylic cycle containing 3 to 8 carbon atoms.

83. a process according to claim 82, wherein the compound with formula (VI) is acetamide, chloracetamide, benzamide, or benzyl carbamate.

84. A process according to claim 82, wherein the amidoalkylation reaction is carried out on the protected phenolic compound in the presence of a protonic acid with a pKa of 4.00 or less, preferably 3.00 or less.

85. A process according to claim 84, wherein the acid is an optionally halogenated mineral oxyacid; a phosphoric acids; a phosphonic acid; an optionally perhalogenated carboxylic acid; or a halogenated sulphonic acid.

86. A process according to claim 82, wherein the reaction is carried out in an organic solvent.

87. A process according to claim 86, wherein the solvent is acetic acid, propionic acid, butyric acid, isobutyric acid, pentanoic acid, or 2-methylbutanoic acid.

88. A process according to claim 82, wherein the ratio between the number of moles of carbonyl compound and the number of moles of protected phenolic compound is at least 1.

89. A process according to claim 88, wherein the ratio between the number of moles of amide or carbamate and the number of moles of carbonyl compound is in the range 1.0 to 3.0.

90. A process according to claim 79, wherein the quantity of strong acid used is such that the mole ratio of H⁺/protected phenolic compound is in the range 1.0 to 20.

91. A process according to claim 82, wherein the reaction is carried out between ambient temperature and 120° C.

92. A process according to claim 53, wherein the amidoalkylated product where the hydroxy function is protected undergoes a treatment using a base employed in a quantity such that the pH is in the range 7 to 10 to cleave the sulphonyl group and liberate the hydroxy group to obtain an amidoalkylated phenolic compound wherein the amidoalkyl group is in the position para to the electron-donating group.

93. A process according to claim 92, wherein the amidoalkylated group is reacted with an acidic solution, to obtain a protected and aminoalkylated phenolic compound wherein the aminoalkyl group is in the position para to the electron-donating group.

94. A process according to claim 93, wherein the protected phenolic compound is reacted with an electrophilic reactant, in the presence of a Friedel-Crafts catalyst.

95. A process according to claim 94, wherein the electrophilic reactant is a carboxylic acid and its halide or anhydride derivatives.

96. A process according to claim 94, wherein the acylation or sulphonylation reactants have the following formulae: 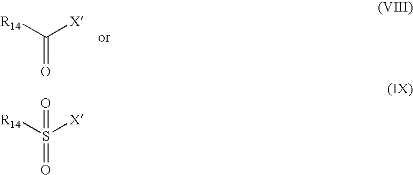
wherein formulae (VIII) or (IX):
R₁₄ represents a linear or branched, saturated or unsaturated aliphatic radical containing 1 to 24 carbon atoms; a saturated, unsaturated or aromatic, monocyclic or polycyclic cycloaliphatic radical containing 4 to 12 carbon atoms; or a linear or branched, saturated or unsaturated aliphatic radical carrying a cyclic substituent;
X′ represents a halogen atom;
in formula (VIII):
X′ represents a —O—CO—R₁₅ radical where R₁₅, which is identical to or different from R₁₄, has the same meaning as R₁₄;
in formula (IX):
X′ represents a —O—SO₂—R₁₅ radical wherein R₁₅, which is identical to or different from R₁₄, has the same meaning as R₁₄;
R₁₄ represents:
an R₁₆—O— alkoxy radical wherein R₁₆ has the same meaning as R₁₄;
an amino group (R₁₇)(R₁₈)—N— wherein R₁₇, which is identical to or different from R₁₈, has the same meaning as R₁₄.

97. A process according to claim 94, wherein the alkylation reactant has general formula (X):
R₁₈—X (X)
wherein:
X represents a hydroxy group or a halogen atom; with the further proviso that said hydroxy group or halogen atom must not be in the vinyl or alkynyl position; and
R₁₈, optionally substituted, represents a linear or branched, saturated or unsaturated acyclic aliphatic radical; a saturated or unsaturated, monocyclic or polycyclic cycloaliphatic radical; or a linear or branched, saturated or unsaturated aliphatic radical carrying a cyclic substituent.

98. A process according to claim 78, wherein the protected phenolic compound is reacted with a carbonyl compound, in the presence of a secondary amine.

99. A process according to claim 98, wherein the carbonyl compound has general formula (XI): 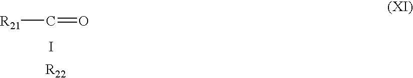
wherein:
R₂, represents a hydrogen atom, a linear or branched alkyl radical containing 1 to 6 carbon atoms, an alkenyl radical containing 2 to 6 carbon atoms, or a phenyl radical optionally substituted; and
R₂₂ is identical to R₂, or represents an electron-attracting group.

100. A process according to claim 98, wherein the secondary amine has the following formula (XII): 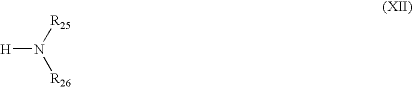
wherein:
R₂₅ and R₂₆, which are identical or different, represent:
a linear alkyl radical containing 1 to 12 carbon atoms, a secondary alkyl radical containing 3 to 12 carbon atoms or a tertiary alkyl radical containing 4 to 12 carbon atoms; the alkyl radicals optionally contain one or two ether functions —O— or hydroxy functions, tertiary amine, carboxylic acid, carboxylic acid ester functions;
a phenylalkyl, cyclohexylalkyl, cycloheptylalkyl or cyclopentylalkyl radical wherein the alkyl portion contains 1 to 4 carbon atoms;
a hydrogen atom;
a phenyl radical, a cyclohexyl radical, a cycloheptyl radical or a cyclopentyl radical;
R₂₅ or R₂₆, together with the nitrogen atom, optionally form a heterocycle saturated or containing one or more double bonds, optionally substituted with one or more alkyl radicals containing 1 to 4 carbon atoms;
R₂₅ and R₂₆ together with the nitrogen atom and with one or more nitrogen or oxygen or sulphur atoms optionally form a saturated or unsaturated heterocycle optionally substituted by one or more alkyl radicals containing 1 to 4 carbon atoms;
R₂₅ and R₂₆ together with the nitrogen atom optionally form an unsaturated heterocycle optionally substituted by one or two methyl or ethyl groups; and
R₂₅ and R₂₆ together, with the nitrogen atom and optionally with one or more nitrogen or oxygen or sulphur atoms optionally form a saturated or unsaturated polycyclic compound optionally substituted by one or more alkyl radicals containing 1 to 4 carbon atoms.

101. A process according to claim 98, wherein the carbonyl compound is reacted, then the secondary amine to form the hemiaminal reactant, then the protected phenolic compound and the acid catalyst.

102. A process according to claim 78, wherein the protected phenolic compound is reacted with a carbonyl compound in the presence of a Brönsted or a zeolite type catalyst.

103. A process according to claim 102, wherein the carbonyl compound has general formula (XI) as defined in claim 45.

104. A process according to claim 78, wherein the protected phenolic compound is reacted with chloral or hexamethylenetetramine.

105. A process according to claim 104, wherein the reaction is carried out in trifluoroacetic acid, sulphuric acid, hydrofluoric acid or boric acid.

106. A process according to claim 95, wherein the electrophilic substitution step is followed by a hydroxy group de-protecting step.
